(19) United States
(12) Patent Application Publication
(10) Pub. No.: US 2009/0299245 A1
(43) Pub. Date: Dec. 3, 2009

(54) Ankle Brace Design for Optimal Fit to Either Right or Left Extremity Contours

(76) Inventor: Gary Blaine Wilkerson, Signal Mountain, TN (US)

Correspondence Address: Gary B. Wilkerson, 9 Big Rock Road, Signal Mountain, TN, 37377, US

(21) Appl. No.: 12/131,989

(22) Filed: Jun. 3, 2008

(51) Int. Cl. A61F 5/00

(52) U.S. Cl. 602/27

(57) ABSTRACT

The invention is a stirrup-type ankle brace that is designed to effectively perform two functions when applied to either a right extremity or a left extremity: 1) dispersal of swelling associated with an ankle sprain, and 2) restraint of excessive joint displacement that would otherwise impose stress on the ankle ligaments. The unique characteristic of the invention is its adaptability to differing anatomical contours on the medial (inner) and lateral (outer) aspects of the right and left extremities, which is achieved through multiple pivoting connections between semi-rigid brace components and adjustable-tension straps, along with symmetrical relationships between all brace components. Thus, the positioning of the moveable and adjustable brace components can be altered to produce either one of two mirror-imaged configurations.

### CROSS REFERENCE TO RELATED APPLICATIONS

This application claims benefit of Provisional application Ser. No. 60/936/665 filed Jun. 22, 2007.

### STATEMENT REGARDING FEDERALLY SPONSORED RESEARCH OR DEVELOPMENT

Not applicable.

### BACKGROUND OF THE INVENTION

The invention relates to restraint of excessive motion of the joints of the ankle and foot for prevention of injury or protection of injured ligaments, as well as application of external compression to body surfaces in a manner that will optimally disperse swelling associated with an ankle ligament sprain. Immediately following injury, control of swelling and joint stabilization provided by a brace can dramatically increase the rate at which normal functional capabilities are restored and the quality of ligament healing that is ultimately realized.

The prior art includes an exceedingly wide variety of ankle brace designs, which have different closure mechanisms, strap configurations, and material characteristics. A simple stirrup-type assembly of semi-rigid elements has been recognized as an effective design for restraint of abnormal ankle displacement and swelling control (Johnson, Jr., U.S. Pat. No. 4,280,489; Burns, U.S. Pat. No. 5,007,416). When subjected to excessive inversion stress, the lateral (outer) articular surfaces of the joints of the foot and ankle are distracted and the medial (inner) articular surfaces are compressed. A stirrup-type ankle brace incorporates elongated medial and lateral panels that are constructed from a relatively rigid plastic. Both components contribute to ankle stability, but have different biomechanical effects. The component that spans the medial joint surfaces acts like a “spacer bar” to resist medial compression. When the lateral component of a stirrup brace exerts pressure against the lateral surface of the ankle and leg, it acts as a “buttress” to resist lateral distraction. Swelling is controlled by resistance to expansion of the soft tissues on the medial and lateral aspects of the ankle. Padding material affixed to the elongated medial and lateral stirrup brace panels provides optimal resistance to soft tissue expansion when it closely conforms to the normal surface contours of the extremity. Straps affixed to the exterior surfaces of the medial and lateral panels are necessary to maintain the positioning of the brace components against the extremity.

Because a wide range of upward and downward foot movement is desirable for activities that involve running and jumping, some stirrup-type ankle brace designs have incorporated hinges between semi-rigid foot and leg components (Westin et al., U.S. Pat. No. 4,646,726; Peters, U.S. Pat. No. 5,031,607; Miklaus et al., U.S. Pat. No. 5,209,722; Wilkerson, U.S. Pat. No. 5,902,259; Quinn et al., U.S. Pat. No. 5,971,946; Peters, U.S. Pat. No. 6,053,884; Richie Jr., U.S. Pat. No. 6,602,215 B1; Morton, U.S. Pat. No. 6,656,145 B1; Bowman, U.S. Pat. No. 6,689,081 B2). Upward and downward movement of the foot results from motion within the talocrural joint, which is widely referred to as the ankle joint, whereas side-to-side movement of the foot results from motion between the talus and calcaneus within the subtalar joint. A joint's “functional” axis of rotation is an imaginary line in space, around which angular motion occurs. The orientation of the functional axis of the talocrural joint closely corresponds to the lower tips of the bony protuberances on either side of the ankle; i.e., the tibial malleolus on the medial aspect and the fibular malleolus on the lateral aspect. In relation to the position of the tibial malleolus, the fibular malleolus is lower and more posterior. The functional axis of the talocrural joint has an oblique spatial orientation; i.e., higher and more anterior on the medial aspect of the ankle; lower and more posterior on the lateral aspect of the ankle.

All stirrup-type ankle brace designs incorporate adjustable-tension straps that secure the medial and lateral semi-rigid panels to the leg. Adjustable-tension straps that connect semi-rigid foot and leg components have also been disclosed (Westin et al., U.S. Pat. No. 4,646,726; Wilkerson, U.S. Pat. No. 5,902,259; Richie Jr., U.S. Pat. No. 6,602,215 B1; Bowman, U.S. Pat. No. 6,689,081 B2). Because abnormal ankle displacement is associated with torque transfer from joints in the forefoot, restriction of excessive forefoot inversion (inward displacement) is essential for optimal maintenance ankle stability (Wilkerson, 2002). To control forefoot inversion, the strap system must span the set of articulations between the talus, calcaneus, navicular, cuboid, and fifth ray. The designs of most hinged ankle support systems reflect a focus on enhancement of the stability of the hindfoot, without any attempt to control motion within the joints of the forefoot.

The extent to which the contours of a semi-rigid ankle brace align with the convex bony protuberances on the medial and lateral surfaces of the ankle is a key factor affecting its comfort and function. Because the fibular malleolus is located in a position that is lower and more posterior on the lateral aspect of the ankle than the position of the tibial malleolus on the medial aspect, many ankle brace designs require separate right and left configurations of the brace elements to ensure optimal fit. As a practical matter, clinicians and administrators in hospital emergency departments, urgent care centers, physician offices, rehabilitation clinics, and athletic training rooms prefer to have a single ankle brace available that can be applied to either a right extremity or a left extremity. Previous ankle brace designs that can be applied to either a right extremity or a left extremity have not provided an optimal alignment between the contours of its semi-rigid components and those of the medial and lateral surfaces of the extremity to which it is applied.

### BRIEF SUMMARY OF THE INVENTION

The incorporation of a double-pivoting bridge component that connects the lateral panel of the ankle brace to its semi-rigid foot component allows for adjustable positioning of the medial and lateral panels to match the positions of the bony protuberances on either extremity, and it provides a means to more closely match the brace's axis of rotation to the ankle's functional axis of rotation (upward and downward motion of the foot in relation to the leg). Furthermore, the further the semi-rigid foot component extends toward the forefoot, the greater the mechanical advantage that is gained for restraint of ankle displacement by an obliquely oriented strap that is attached to its anterior-lateral portion. The double-pivoting connector bridge component provides a mechanism to effectively create greater footplate length; i.e., posterior positioning of the lateral panel allows for a shorter portion of the footplate to lie between the fibular malleolus and the posterior edge of the footplate component, with a longer portion extending from the fibular malleolus toward the anterior edge of the forefoot. This provides the means to make the foot component longer than that used by any previous brace designed to fit either a right or left extremity.

To function properly in restricting ankle motion, a stirrup-type ankle brace must be secured to the lower leg by means of straps crossing the anterior and posterior aspects of the leg. Slots cut into the semi-rigid plastic material comprising the stirrup upright components can serve as anchor points for the straps, and they maintain the positions of the leg straps in a manner similar to the function served by belt loops on trousers. The precise shape of slots on the anterior and posterior margins of the medial and lateral brace panels determines the extent to which a strap assumes an orientation that conforms to the leg contours on the anterior and posterior aspects of the leg. Curvature in the shape of the lower portion of the slots allows the orientation of the strap threaded through them to assume an oblique orientation when encasing the posterior aspect of the leg. A vertical orientation of the upper portion of the slots allows the orientation of the strap threaded through them to assume a horizontal orientation when encasing the anterior aspect of the leg. Thus, the “J-shaped” slot configuration is a unique design feature that enhances conformity of the ankle brace to either a right or left extremity.

Another design feature that facilitates optimal brace function on either a right or left extremity is the exact location of pivoting strap fixation hardware (e.g., D-rings) on the medial leg panel, which determines the relative lengths of two separate crisscrossing straps that originate on the anterior-lateral and posterior-lateral margins of the footplate component. The pivoting connection of the strap fixation hardware to the medial panel maintains approximately equal length of the two crisscrossing straps when the direction of their respective functions are reversed by a change in the brace's configuration for application to the opposite extremity (right-to-left or left-to-right).

The integrated function of the double-pivoting bridge component, curved leg strap slots, and pivoting fixation hardware for the crisscrossing straps collectively optimize the ankle stabilization effect, swelling dispersal effect, and comfort of a stirrup-type ankle brace that can be applied to either a right extremity or a left extremity.

### BRIEF DESCRIPTION OF THE DRAWINGS

FIG. 1 is a medial side view of the invention applied to a right ankle.

FIG. 2 is a lateral side view of the invention applied to a right ankle.

FIG. 3 is a lateral side view of the invention applied to a right ankle, with a portion of the crisscrossing strap system removed for full visualization of the double-pivoting bridge connector between the footplate and lateral panel components.

FIG. 4 is a depiction of the relationship between the tibial malleolus and the fibular malleolus, and the approximate orientation of the functional axis of the talocrural joint, for left and right extremities.

FIG. 5 provides views of the undersurface of the footplate component of the invention when configured for application to a right extremity (A) and configured for application to a left extremity (B).

FIG. 6 provides lateral side views of the invention when configured for application to a right extremity (A) and configured for application to a left extremity (B).

FIG. 7 provides medial side views of the invention when configured for application to a right extremity (A) and configured for application to a left extremity (B).

### DETAILED DESCRIPTION OF THE INVENTION

The invention incorporates a semi-rigid foot component 1 that consists of a flat footplate beneath the foot and perpendicular upright portions on its medial and lateral sides. The foot component 1 articulates with a semi-rigid medial leg panel 2 and a double-pivoting connector bridge 3 that joins the foot component 1 with a semi-rigid lateral leg panel 4. Thus, there is a single pivot point created on the medial side by rivet 5 and two pivot points created by rivet 6 and rivet 7 on the lateral side. A cushioning pad 8, which has the same general shape as that of the medial panel, is affixed to the undersurface of the medial panel. A removable U-shaped pad 9 is affixed to the undersurface of the lateral panel, which provides optimal compression of the soft tissues on the periphery of the fibular malleolus when the brace is used as a therapeutic orthosis. Alternatively, a cushioning pad that has the same general shape as that of the lateral panel may be used when control of ankle swelling is not a concern. An oval-shaped opening 24 in the semi-rigid lateral panel 4 provides a means to confirm proper placement of the brace over the fibular malleolus.

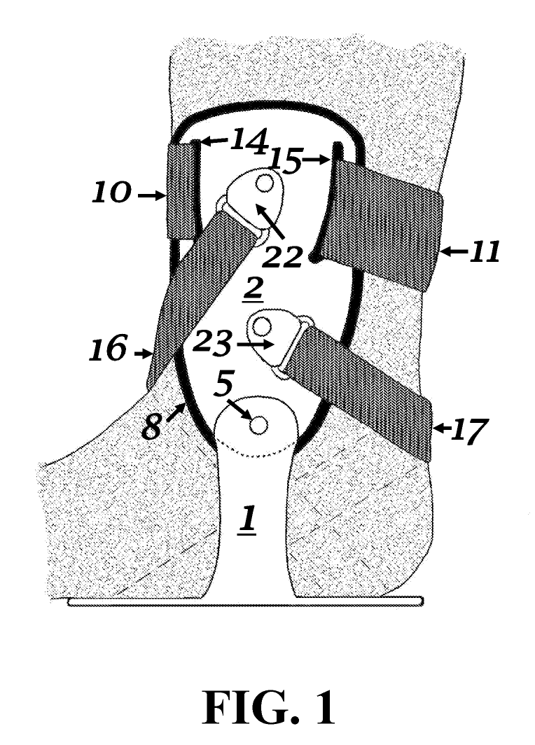

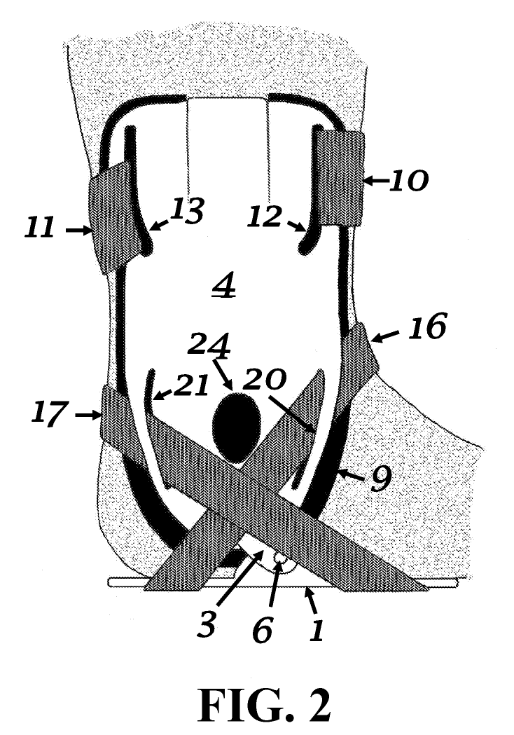

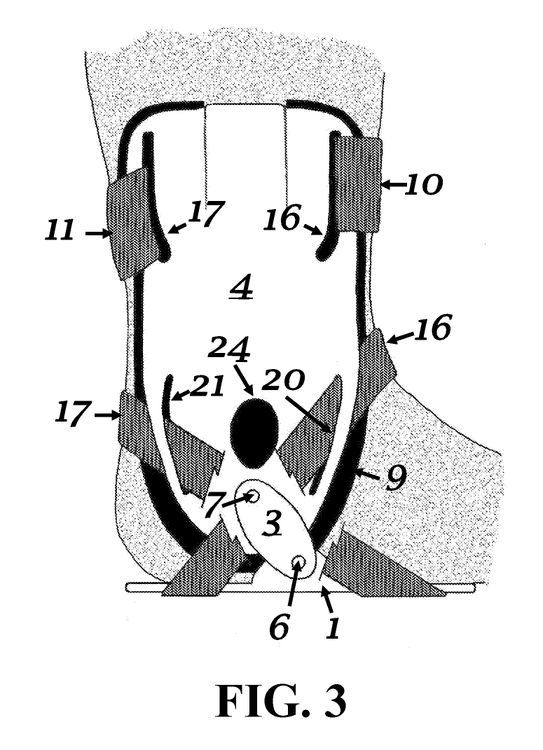

Straps 10 and 16, which both cross the anterior aspect of the leg when the brace is applied to a right extremity, and slots 12 and 20 on the anterior margin of the lateral panel and slot 14 on the anterior margin of the medial panel, are identified by even numbers. Corresponding straps that cross the posterior aspect of the leg and slots on the posterior margins of the lateral and medial panels are identified by odd numbers. The first end of strap 10 is affixed to itself by means of a Velcro hook tab that adheres to Velcro loop material on undersurface of the strap. Assuming that the brace is configured for application to a right extremity (FIGS. 1-3), strap 10 passes through the upper portion of slot 12 on the anterior margin of the lateral panel, crosses the anterior aspect of the leg, passes through the upper portion of slot 14 on the anterior margin of the medial panel, and is again affixed to itself by means of a Velcro hook tab that adheres to loop material on the outer surface of strap 10. Strap 11 is secured at both ends in the same manner as strap 10. It passes through the lower curved portion of slot 13 on the posterior margin of the lateral panel, crosses the posterior aspect of the leg at the base of the calf musculature, and passes through the lower curved portion of slot 15 on the medial panel. Strap 16 originates from pivoting rivet fixation point 18 on the posterior-lateral portion of the semi-rigid footplate 1. It follows an oblique course across the outer surface of the lateral panel 4, passes through slot 20, crosses the anterior aspect of the leg, passes through pivoting buckle assembly 22, and its end is fastened to Velcro loop material on the outer surface of the strap by means of a Velcro hook tab at the end of the strap. Strap 17 originates from pivoting rivet fixation point 19 on the posterior-lateral portion of the semi-rigid footplate 1. It follows an oblique course across the outer surface of the lateral panel 4, passes through slot 21, crosses the anterior aspect of the leg, passes through pivoting buckle assembly 23, and its end is fastened on the outer surface of the strap by means of Velcro hook and loop material.

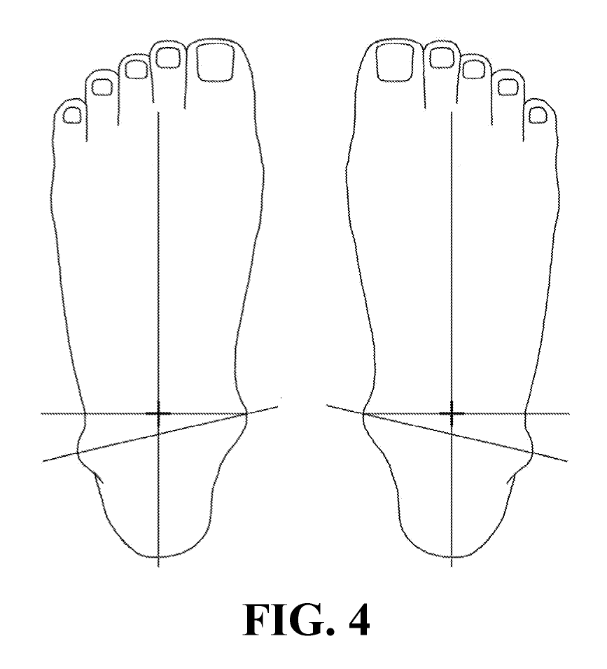

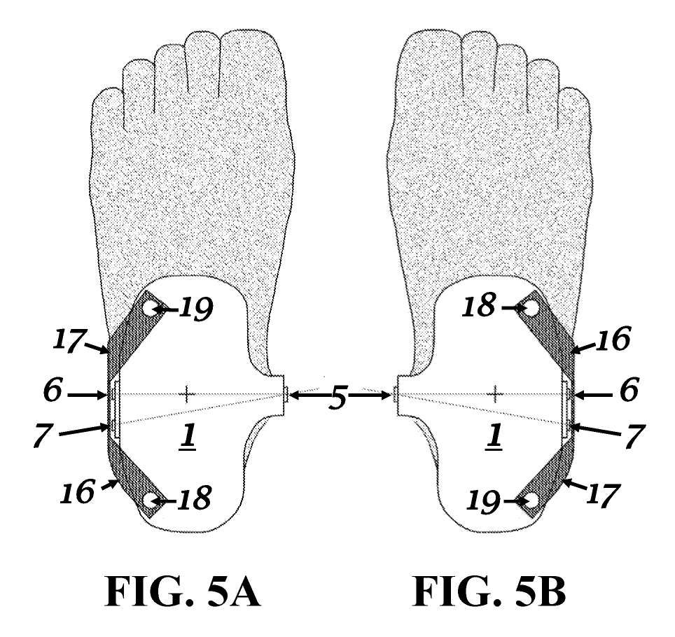

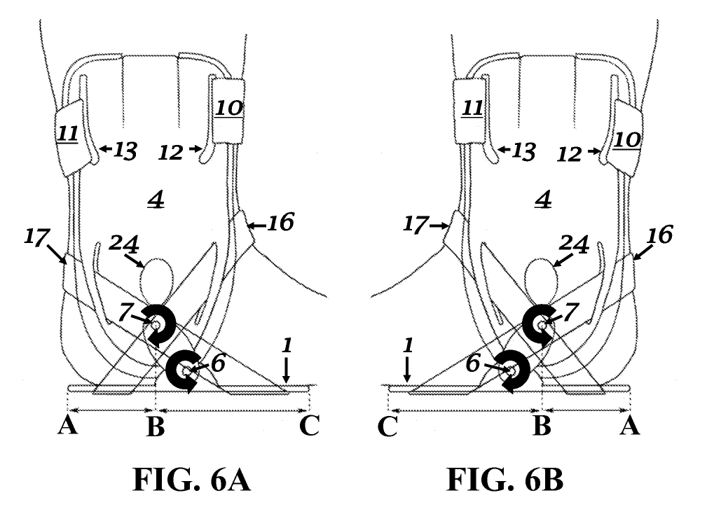

The unique characteristic of the invention is the adaptability of its components to accommodate the opposite spatial locations of corresponding convex bony protuberances on the right and left extremities (FIGS. 4-7). When the brace is applied to a left extremity, the numbering convention is reversed, i.e., the odd-numbered components have more anterior positions on the lateral and medial panels, and the even-numbered components have more posterior positions. Opposite directions of rotation of pivot points 6 and 7 on bridge connector 3 convert the brace configuration to accommodate the anatomical contours of an opposite extremity (FIG. 6: right-to-left or left-to-right conversion). The strap that crosses the anterior aspect of the leg (10 or 11) conforms to its surface contours in a horizontal position, but the strap that crosses the posterior aspect of the leg (10 or 11) must assume a relative oblique orientation to optimally conform to the surface contours created by the calf musculature. This differing strap orientation is accommodated by the curvature of slots 12 and 13 on the lateral panel 4 and slots 14 and 15 on medial panel 2.

The straps anchored beneath footplate component 1 have oppositely-directed oblique courses that crisscross one another (16 and 17) over the lateral panel 4. The double-pivoting connector bridge component 3 provides a mechanism to position the lateral panel 4 over the posterior portion of the footplate component 1, which increases the mechanical advantage of the oblique strap anchored at the anterior-lateral margin of footplate 1 (16 or 17) for restraint of inward forefoot displacement; i.e., posterior positioning of lateral panel 4 in relation to the midpoint of the footplate 1 increases the distance between the fibula and the anterior margin of the footplate (FIG. 6 line BC).

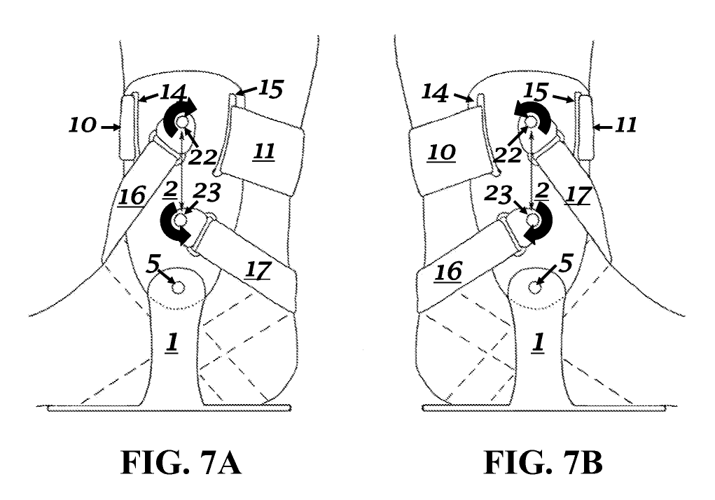

The strap anchored to footplate 1 at its anterior-lateral margin (17 in FIGS. 6A and 16 in FIG. 6B) crosses the posterior aspect of the leg, whereas the strap anchored to the footplate at its posterior-lateral margin (16 in FIGS. 6A and 17 in FIG. 6B) crosses the anterior aspect of the leg. Because the strap anchored to the anterior-lateral margin of footplate 1 spans a longer distance across the lateral aspect of the foot and ankle, and has a lower position as it crosses the posterior aspect of the leg to the medial panel 2, a low attachment point on medial panel 2 better accommodates the strap's course and length than a high location (17 in FIGS. 7A and 16 in FIG. 7B). Because the strap anchored to the posterior-lateral margin of footplate 1 spans a shorter distance across the lateral aspect of the foot and ankle, and has a higher position as it crosses the anterior aspect of the leg to the medial panel 2, a high attachment point on medial panel 2 better accommodates the strap's course and length than a low location. The pivoting fixation points of both strap buckle assemblies 22 and 23 are located along the vertical midline of the medial panel 2. When the brace is applied to a right extremity (FIG. 7A), strap 17 passes behind the leg and connects to the lower strap buckle assembly 23, and strap 16 passes across the front of the leg and connects to the higher strap buckle assembly 22. When the brace is applied to a left extremity (FIG. 7B), strap 16 passes behind the leg and connects to the lower strap buckle assembly 23, and strap 17 passes across the front of the leg and connects to the higher strap buckle assembly 22.

What is claimed is:

1. A stirrup-type ankle brace that optimizes congruency between the contours of its structure and the opposite anatomical contours of either a right or left extremity, which includes a semi-rigid foot component that articulates with a semi-rigid medial leg panel and a double-pivoting semi-rigid connector bridge that joins the foot component to a semi-rigid lateral leg panel.

2. The stirrup-type ankle brace of claim 1, wherein slots in the semi-rigid medial and lateral panels for attachment of anterior and posterior leg straps have a curvature toward the midline of each panel at their lower ends, which optimizes strap conformity to the anatomical contours of either a right or left extremity.

3. The stirrup-type ankle brace of claim 1, wherein the semi-rigid medial panel incorporates two pivoting strap buckle assemblies that are positioned at the midline of the panel to receive straps anchored to the anterior-lateral and posterior-lateral margins of the foot component in a manner that permits reversal in the function of the straps and buckle assemblies for application to either a right or left extremity.
